(19) United States
(12) Patent Application Publication
(10) Pub. No.: US 2013/0324828 A1
(43) Pub. Date: Dec. 5, 2013

(54) BIOLOGICAL ELECTRODE AND ROLL OF BIOLOGICAL ELECTRODE

(71) Applicant: NIHON KOHDEN CORPORATION, Tokyo (JP)

(72) Inventors: Shigehiro NISHIWAKI, Tokyo (JP); Kouichiro MINAMI, Kawasaki-shi (JP)

(73) Assignee: NIHON KOHDEN CORPORATION, Tokyo (JP)

(21) Appl. No.: 13/903,290

(22) Filed: May 28, 2013

(30) Foreign Application Priority Data
Jun. 5, 2012 (JP) 2012-128023

(51) Int. Cl. A61B 5/0408

(52) U.S. Cl. CPC A61B 5/04085
USPC 600/391; 600/393

(57) ABSTRACT

A biological electrode includes: a substrate which is insulative and flexible; a plurality of electrode terminals which are arranged on a first surface of the substrate, and which are separated from each other at equal intervals and are arranged in one row; and a plurality of electrically conductive members which are arranged on first portions of a second surface of the substrate, and which are not electrically conductive to each other, the plurality of electrically conductive members corresponding to the plurality of electrode terminals, the electrically conductive members each of which is connected to a corresponding one of the electrode terminals and is adapted to be electrically contactable to a living body. The substrate is formed with first separating portions between respective adjacent pairs of sets of one of the electrode terminals and a corresponding one of the electrically conductive members.

### CROSS-REFERENCE TO RELATED APPLICATION(S)

This application is based upon and claims the benefit of priority from prior Japanese patent application No. 2012-128023, filed on Jun. 5, 2012, the entire contents of which are incorporated herein by reference.

### BACKGROUND

The presently disclosed subject matter relates to a biological electrode and a roll of biological electrode.

In a clinical site, it is requested to rapidly measure biological information of a patient such as an electrocardiogram. In a biological information measuring apparatus such as an electrocardiograph, usually, an electric signal which is produced in a living body in accordance with a change of the state of the living body is detected through electrodes applied to the skin surface of the living body, and the electric signal is analyzed to acquire biological information such as an electrocardiogram. A medical person such as a doctor or a paramedic diagnoses the condition of the patient with reference to the acquired biological information, and provides necessary measures to the patient. In an emergency medical site where a medical person is requested to promptly respond to the patient, particularly, a plurality of electrodes must be applied for a short time period to predetermined positions of the body surface of the patient to which the electrodes are to be applied (hereinafter, such positions are referred to as electrode application positions).

In an emergency medical site, a method has been employed where electrodes which were produced one by one, and which were packed into bags each containing a predetermined number of electrodes are applied one by one to the electrode application positions of the body surface of the patient. However, the method has a problem in that electrodes are applied one by one to the electrode application positions, and therefore the method is cumbersome and requires a long time period. From the viewpoint that a plurality of electrodes are applied easily and rapidly, by contrast, JP-A-2007-50033 discloses a sheet-like electrocardiograph electrode in which three electrodes are placed at the apexes of a triangular, respectively. In the technique disclosed in JP-A-2007-50033, the electrocardiograph electrode which is in the original sheet-like state is applied to the patient, the electrodes are then separated from each other and rearranged, and thereafter an electrocardiogram is measured by the three-electrode lead method.

In the electrocardiograph electrode having the structure disclosed in JP-A-2007-50033, however, it is assumed that an electrocardiogram is measured by the three-electrode lead method, and an electrocardiogram which requires a larger number of electrodes, such as a 12-lead electrocardiogram cannot be measured.

### SUMMARY

The presently disclosed subject matter may provide a biological electrode which can be used in an electrocardiogram measurement requiring a large number of electrodes, and which can be applied to electrode application positions for a short time period.

The biological electrode may comprise: a substrate which is insulative and flexible; a plurality of electrode terminals which are arranged on a first surface of the substrate, and which are separated from each other at equal intervals and are arranged in one row; and a plurality of electrically conductive members which are arranged on first portions of a second surface of the substrate, and which are not electrically conductive to each other, the plurality of electrically conductive members corresponding to the plurality of electrode terminals, the electrically conductive members each of which is connected to a corresponding one of the electrode terminals and is adapted to be electrically contactable to a living body, wherein the substrate is formed with first separating portions between respective adjacent pairs of sets of one of the electrode terminals and a corresponding one of the electrically conductive members.

The biological electrode may further comprise adhesive members used for causing the biological electrode to adhere to the living body, which are arranged on second portions of the second surface of the substrate, and which are formed with second separating portions at positions corresponding to positions of the first separating portions.

The substrate may be formed with notches at positions where long sides of the substrate intersect with the first separating portions, respectively.

The interval between adjacent two of the electrode terminals may be 3 to 8 cm.

There is also provided a roll of biological electrode which includes the biological electrode which is wound in roll form.

### BRIEF DESCRIPTION OF THE DRAWINGS

FIG. 1A is a plan view showing the front surface of a biological electrode of a first embodiment of the presently disclosed subject matter.

FIG. 1B is a plan view showing the back surface of the biological electrode shown in FIG. 1A.

FIG. 1C is a sectional view of the biological electrode shown in FIG. 1A taken along line C-C.

FIG. 2 is a view illustrating an example of packing of the biological electrodes of the first embodiment.

FIG. 3 is a partial diagram of the human body illustrating electrode application positions for biological electrodes.

FIG. 4 is a partial diagram of the human body illustrating electrode application positions for biological electrodes in the case where biological electrodes are to be used in Holter electrocardiography.

FIG. 5A is a view illustrating a case where, in the first embodiment, the biological electrodes are to be used in measurement of chest leads of a 12-lead electrocardiogram.

FIG. 5B is a view illustrating a case where, in the first embodiment, the biological electrodes are to be used in measurement of chest leads and four limb leads of a 12-lead electrocardiogram.

FIG. 5C is a view illustrating a case where, in the first embodiment, the biological electrodes are to be used in the three-electrode method (three-lead method).

FIG. 5D is a view illustrating a case where, in the first embodiment, the biological electrodes are to be used in a five-electrode electrocardiogram measurement.

FIG. 5E is a view illustrating a case where, in the first embodiment, the biological electrodes are to be used in a six-electrode electrocardiogram measurement.

FIG. 6A is a view showing execution time periods when experiments in which the biological electrode of the first embodiment was applied to the subject were executed.

FIG. 6B is a view showing execution time periods when experiments in which related-art biological electrodes were applied to the subject were executed, as comparison examples.

FIG. 7 is a view illustrating a roll of biological electrode of a second embodiment of the presently disclosed subject matter.

### DETAILED DESCRIPTION OF EXEMPLARY EMBODIMENTS

Hereinafter, an embodiment of the biological electrode of the presently disclosed subject matter will be described with reference to the accompanying drawings. In the figures, the identical components are denoted by the same reference numerals. In the drawings, the dimension ratios are exaggerated for the sake of convenience in description, and may be sometimes different from the actual ratios.

### First Embodiment

<Structure of Biological Electrode>

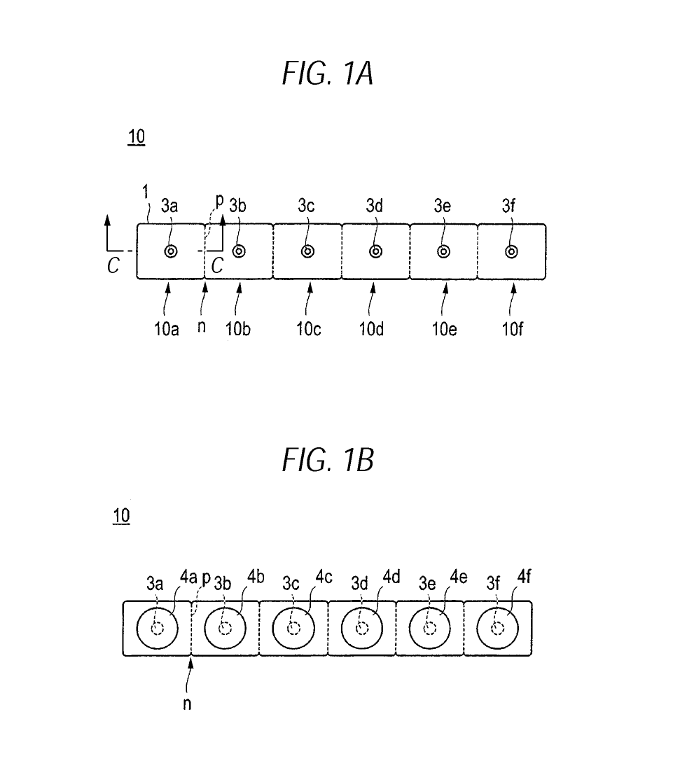

FIG. 1A is a plan view showing the front surface of a biological electrode of a first embodiment of the presently disclosed subject matter, FIG. 1B is a plan view showing the back surface of the biological electrode shown in FIG. 1A (a state where a release sheet 2 which will be described later is peeled off), and FIG. 1C is a sectional view of the biological electrode shown in FIG. 1A taken along line C-C. Hereinafter, a case where the biological electrode of the embodiment is used in an electrocardiogram measurement will be exemplarily described. However, the biological electrode of the embodiment maybe used also in a measurement of other biological information.

As shown in FIGS. 1A to 1C, the biological electrode 10 of the embodiment has a substrate 1, the release sheet 2, electrode terminals 3a to 3f, and electrically conductive members 4a to 4f. Clips, which are formed by an electrically conductive material and which are used for transmitting electrocardiographic signals to a body unit of an electrocardiograph that is not shown, are connectable to the electrode terminals 3a to 3f respectively. The conductive members 4a to 4f are formed by a conductive gel, and placed on the back surface of the substrate 1 while being contacted with the electrode terminals 3a to 3f to be electrically conductive to the electrode terminals 3a to 3f, respectively, so that, when the biological electrode 10 is attached to the patient, the terminals are electrically connected to the body surface of the patient.

The substrate 1 is a strip-like sheet member which is insulative and flexible, and which is formed by a material such as a resin. In middle portions in the width directions of the substrate 1, through holes for attaching the electrode terminals 3a to 3f are disposed respectively along the longitudinal direction at equal intervals. In the substrate 1, moreover, a perforation line p (separating portion) which is perpendicular to the longitudinal direction is formed in a substantially middle portion between two adjacent electrode terminals.

In the embodiment, the substrate 1 is formed into a substantially rectangular shape, and a non-conductive adhesive member AD for adhesively fixing the substrate 1 to the body surface of the patient which is a living body is bonded to a portion of the back surface of the substrate 1 excluding the conductive members 4a to 4f. The adhesive member AD and the conductive members 4a to 4f are covered by the release sheet 2. Also in the adhesive member AD and the release sheet 2, perforation lines are formed at the same positions as those of the perforation lines p of the substrate 1.

In the substrate 1, the adhesive member AD, and the release sheet 2, notches n are formed at positions where the long sides of the substrate 1 intersect with the perforation lines.

In a region between two adjacent electrode terminals, with using the perforation line and the notches n, a medical person can divide the biological electrode 10 into a plurality of portions. More specifically, for example, a medical person can cut off an end portion of the substrate 1 along the perforation line between the electrode terminals 3a and 3b by the fingers. In other words, it is possible to easily separate a unit electrode 10a including the substrate 1 to which the electrode terminal 3a is attached. When the biological electrode 10 is to be used, the medical person peels the release sheet 2 from the back surface of the substrate 1, and then applies the electrode to the body surface of the patient.

In the embodiment, the biological electrode 10 includes six unit electrodes 10a to 10f. In order to use the biological electrode in chest leads which are most frequently employed, preferably, the biological electrode 10 includes six or more unit electrodes.

FIG. 1C shows the unit electrode 10a. The electrode terminal 3a has a base portion BS, a projection PR which is projected from the base portion BS, and a cap CT which is fitted to the projection PR. The cap CT is fitted to the projection PR in a state where the cap cooperates with the base portion BS to clamp the substrate 1. A clip which is used for connecting a signal line cable that extends from the body unit of the electrocardiograph, and that is not shown can be connected to the cap CT. The portion of the base portion BS which is contacted with the conductive member 4a is covered by a covering layer of Ag/AgCl. The same structure is applicable also to the unit electrodes 10b to 10f.

As shown in FIGS. 1A and 1B, the electrode terminals 3a to 3f are arranged in one row on the substrate 1 while being separated at equal intervals. The caps CT fitted to the projections PR which are passed through the through holes of the substrate 1 are located on the front surface of the substrate 1, and the base portions BS are located on the back surface of the substrate 1. In the embodiment, the intervals of adjacent electrodes are set to about 5 cm in view of the standard body dimension of an adult male. When considering various body dimensions, it is preferred to prepare electrodes having intervals ranging from 3 cm to 8 cm in steps of 1 cm. The electrode terminals 3a to 3f are electrically connectable to the body unit of the electrocardiograph through the signal line cable. In the signal line cable, clips (not shown) which are to clampingly hold the projections PR are disposed so that electrical connections with the electrode terminals 3a to 3f can be ensured.

The conductive members 4a to 4f are to be contacted with the skin surface of the patient to ensure electrical connections between the skin surface of the patient and the electrode terminals 3a to 3f.

The thus configured biological electrode 10 of the embodiment has: the electrode terminals 3a to 3f which are arranged in one row on the substrate 1 while being separated at equal intervals; and the conductive members 4a to 4f which are to be contacted with the skin surface of the patient to ensure electrical connections between the skin surface of the patient and the electrode terminals 3a to 3f.

<Example of Packing of Biological Electrodes>

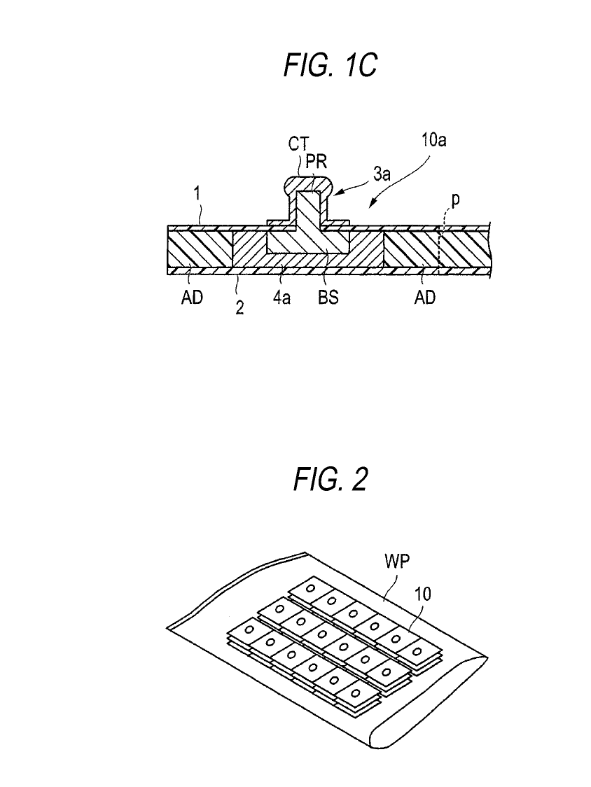

Next, packing of the biological electrodes of the embodiment will be described with reference to FIG. 2. FIG. 2 is a view illustrating an example of packing of the biological electrodes of the embodiment.

In the embodiment, the biological electrodes 10 are accommodated in a packing material WP in a state where bundles of a predetermined number of biological electrodes are laterally arranged. For example, the packing material WP is a polyvinyl bag. FIG. 2 exemplarily shows a case where three bundles each consisting of three biological electrodes are laterally juxtaposed in three rows. In the biological electrode 10 of the embodiment, the unit electrodes are continuously formed in one row. Therefore, the biological electrodes 10 can be packed while the biological electrodes are aligned and overlapped with each other. As compared with the case where biological electrodes are produced one by one and a predetermined number of biological electrodes are separately packed in the packing material WP, consequently, extra gap spaces between the electrodes in the packing material WP are reduced, and hence the volume occupied by the biological electrodes 10 in the packing material WP can be lessened. As a result, the accommodation property of the biological electrodes 10 is improved.

<Method of Applying Biological Electrode>

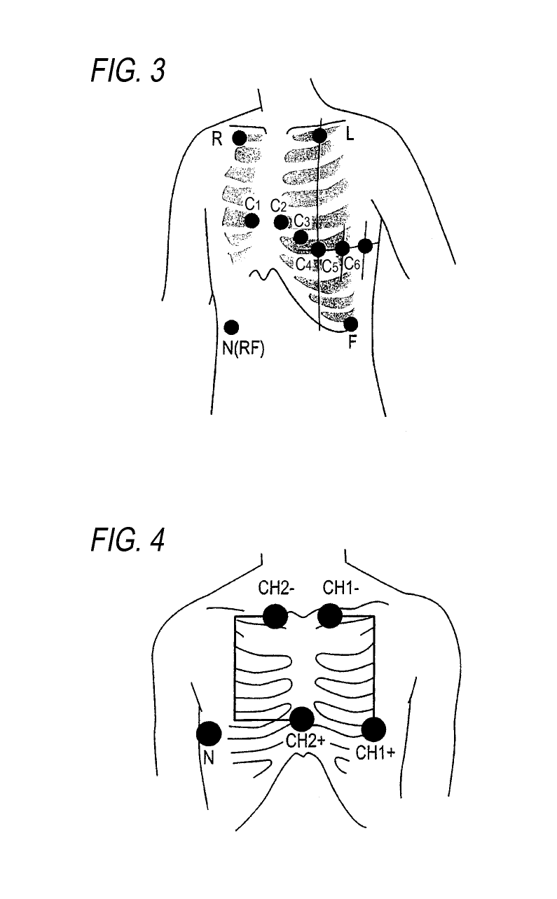

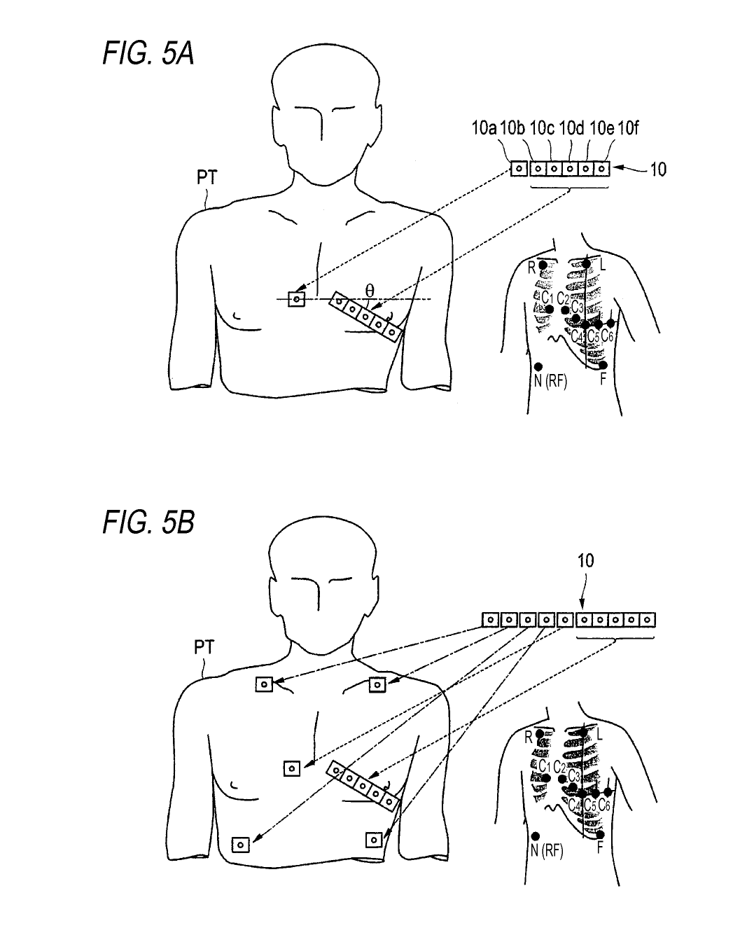

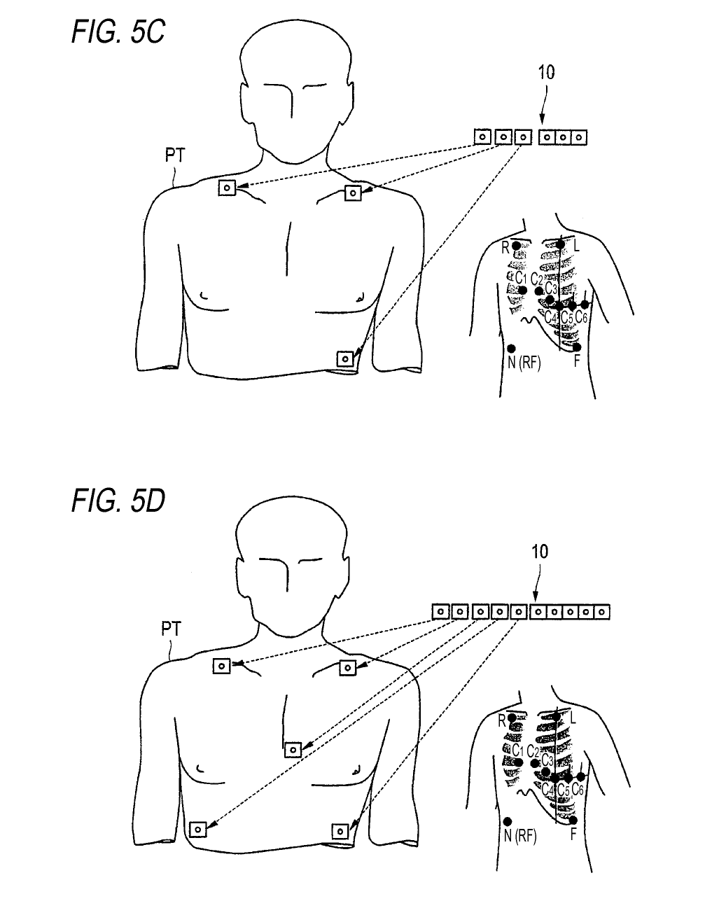

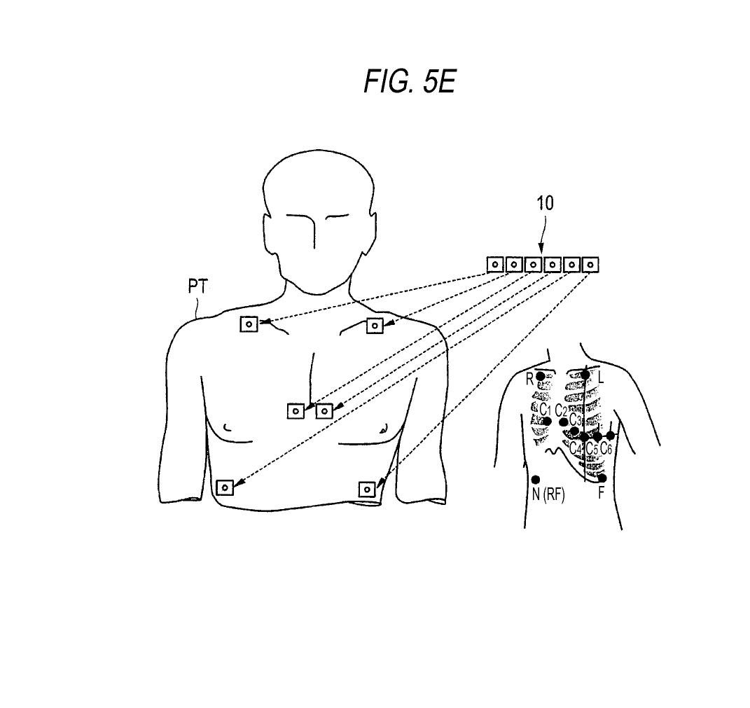

Next, a method of applying the biological electrode of the embodiment will be described with reference to FIGS. 3, 4, and 5A to 5E. FIG. 3 is a partial diagram of the human body illustrating electrode application positions for biological electrodes, and FIG. 4 is a partial diagram of the human body illustrating electrode application positions for biological electrodes in the case where biological electrodes are to be used in Holter electrocardiography. FIG. 5A is a view illustrating a case where, in the embodiment, the biological electrode is to be used in measurement of chest leads of a 12-lead electrocardiogram, FIG. 5B is a view illustrating a case where, in the embodiment, the biological electrode is to be used in measurement of chest leads and four limb leads of a 12-lead electrocardiogram, FIG. 5C is a view illustrating a case where, in the embodiment, the biological electrode is to be used in monitoring of the three-electrode method (three-lead method), FIG. 5D is a view illustrating a case where, in the embodiment, the biological electrode is to be used in a five-electrode electrocardiogram measurement, and FIG. 5E is a view illustrating a case where, in the embodiment, the biological electrode is to be used in a six-electrode electrocardiogram measurement.

In measurement of a 12-lead electrocardiogram, as shown in FIG. 3, electrodes are applied to electrode application positions C1 to C6, R, L, F, and N(RF). The electrode application positions C1 and C2 are bilaterally symmetrical with respect to the stermum, and C2 to C6 are located in a substantially straight line. Electrodes which are to be used in chest leads V1 to V6 are applied to the positions C1 to C6, respectively. Electrodes which are to be used in four limb leads are applied to the positions R, L, F, and N(RF), respectively. In the case where a 12-lead electrocardiogram of chest leads and four limb leads is to be measured, a medical person applies electrodes to the electrode application positions C1 to C6, R, L, F, and N(RF), and attaches the signal line cable extending from the electrocardiograph to the respective electrode terminals of the electrodes. In a 12-lead electrocardiogram, ten electrodes are applied to the body surface of the patient as described above, and leads in a total of 12 directions configured by chest leads and four limb leads are recorded.

In measurement of the three-electrode method, electrodes are applied to the positions R, L, and F. The three-electrode method is usually called a monitor electrocardiogram, and used in the case where an electrocardiogram is to be monitored in a patient room (bedside monitor) and a nurse station (central monitor).

In an electrocardiogram measurement in the case where five electrodes are used, the electrodes are applied to the positions R, L, F, and N(RF), and any one of C1 and C26 In an electrocardiogram measurement in the case where six electrodes are used, the electrodes are applied to the positions R, L, F, and N(RF), and any two of C1 to C6. In an electrocardiogram measurement in the case where five electrodes or six electrodes are used, a part of chest leads can be measured in addition to four limb leads of a 12-lead electrocardiogram.

In the case where the electrodes are to be used in Holter electrocardiography, as shown in FIG. 4, the electrodes are applied to electrode application positions CH1+, CH1−, CH2+, CH2−, and N. A Holter electrocardiograph is produced in small size so that it can be carried by the patient. When electrodes are applied to the body surface of the patient for a time period which is longer than that in a usual electrocardiogram measurement, the electrocardiograph can measure a long-term electrocardiogram.

In the case where the biological electrode 10 of the embodiment is to be used in measurement of chest leads of a 12-lead electrocardiogram, as shown in FIG. 5A, a medical person separates the unit electrode 10a from the body of the biological electrode 10, and applies the unit electrode to the position C1 of the patient PT. Then, the medical person applies the biological electrode 10 so that the unit electrode 10b corresponds to the position C2, and the unit electrode 10f corresponds to the position C6. Namely, the unit electrodes 10b to 10f are linearly applied while the position C2 is set as a starting point, and the position C6 is set as an ending point. At this time, preferably, the angle θ formed by the line connecting C1 and C2 and the biological electrode 10 is about 15 deg.

When the biological electrode 10 is linearly applied while the position C1 is set as a starting point, and the position C6 is set as an ending point, the position where the unit electrode 10b is actually applied is downward deviated from the position C2. Therefore, the unit electrode 10a is separated from the body of the biological electrode 10. When ischemia of the left main coronary trunk is to be determined, the chest lead V2 must be correctly measured. Therefore, the unit electrode 10b must be correctly applied to the position C2.

In order that, in emergency care, a 12-lead electrocardiogram is measured and localized anterior, anteroseptal, or anterolateral infarction is diagnosed, it is necessary to measure chest leads. Particularly, it is important to correctly apply the unit electrodes 10a, 10b to the positions C1, C2. In the embodiment, therefore, the unit electrode 10a is separated from the body of the biological electrode 10 and applied to the position C1, and the unit electrodes 10b to 10f are linearly applied while the position C2 is set as a starting point, and the position C6 is set as an ending point.

In the case where the biological electrode of the embodiment is to be used in measurement of chest leads and four limb leads of a 12-lead electrocardiogram, the biological electrode 10 including ten unit electrodes is used as shown in FIG. 5B. In the ten unit electrodes, four unit electrodes for four limb leads are separated, and then applied to the positions R, L, F, and N(RF), respectively. The remaining six unit electrodes are applied to C1 to C6 in accordance with the above-described method of applying biological electrodes in chest leads.

In the embodiment, when six unit electrodes which are to be used in measurement of chest leads V1 to V6 are to be applied, as described above, only one unit electrode which is to be applied to the electrode application position C1 is separated, and the remaining unit electrodes are linearly applied so as to correspond to the electrode application positions C2 to C6 of the chest of the patient PT. As compared with the case where biological electrodes which are produced one by one are applied respectively to the electrode application positions C2 to C6 of the chest of the patient PT, therefore, the applying work is not cumbersome. As a result, the biological electrode can be used also in a measurement requiring a large number of electrodes, such as a 12-lead electrocardiogram, and applied to electrode application positions of the chest of the patient PT for a short time period. Since the intervals of adjacent electrodes are constant, the electrode terminals 3b to 3f corresponding to chest leads V2 to V6 can be applied to predetermined positions. Therefore, it is possible to prevent the position where the biological electrode 10 is applied, from being dispersed among medical persons who perform an applying work.

Moreover, the unit electrodes of the biological electrode 10 of the embodiment are configured so as to be separated from each other, and hence can be used also in the case of monitoring of the three-electrode method (three-lead method), a five-electrode measurement, Holter electrocardiography, a six-electrode measurement, or the like.

In the case where the biological electrode 10 of the embodiment is to be used in monitoring of the three-electrode method as shown in FIG. 5C, for example, a biological electrode including six unit electrodes is used, and three unit electrodes 10a, 10b, 10c are separated, and applied to the electrode application positions R, L, and F, respectively. The remaining three electrodes are stored for the next use.

In the case where the biological electrode is to be used in a five-electrode electrocardiogram measurement as shown in FIG. 5D, the biological electrode 10 including ten unit electrodes is used. In the ten unit electrodes, five unit electrodes are separated. Among the five unit electrodes, four unit electrodes are applied to the positions R, L, F, and N(RF), respectively. The remaining one unit electrode is applied to any one of the electrode application positions C1 to C6. In a five-electrode electrocardiogram, myocardial ischemia monitoring can be performed. In a measurement for Holter electrocardiography, unit electrodes are applied to the electrode application positions CH1+, CH1−, CH2+, CH2−, and N shown in FIG. 4. The remaining five electrodes which have not been separated are stored for the next use.

In the case where the biological electrode is to be used in a six-electrode electrocardiogram measurement as shown in FIG. 5E, the biological electrode 10 including six unit electrodes is used. Six unit electrodes are separated, and then are applied to the positions R, L, F, and N(RF), and any two of C1 to C6, respectively.

In the above, the cases where the number of unit electrodes included in the biological electrode is six or ten have been exemplarily described. However, the number of unit electrodes included in the biological electrode is not limited to six or ten. For example, the number of unit electrodes included in the biological electrode maybe three to five. When the number of unit electrodes is seven, eight, nine, or ten or more, the biological electrode can cope with a patient with a large body frame. In this case, electrode terminals of unit electrodes which are remote from the electrode application positions, and which are not used in measurement may not be connected to the signal line cable.

When a biological electrode in which the number of unit electrodes is increased, and the intervals of adjacent electrode terminals are narrowed is used, the unit electrodes can be applied to the most appropriate positions. In the case where the biological electrode is to be applied to C2 to C6, for example, it is sufficient to prepare at least five unit electrodes. When a biological electrode in which the number of unit electrodes is increased to, for example, seven and the intervals of adjacent electrode terminals are narrowed is used, however, the unit electrodes can be applied to more correct positions. The biological electrode can cope also with this case by not connecting electrode terminals which are remote from the electrode application positions, and which are not used in measurement, to the signal line cable. Particularly, the intervals between C3 and C4, and C4 and C5 often vary between individual. In this case, therefore, the use of a biological electrode in which the intervals of adjacent electrode terminals are narrowed exerts a large effect. When a biological electrode in which the intervals of adjacent electrode terminals are narrowed is additionally prepared, the biological electrode can cope with a patient with a small body frame such as a child. When several types of biological electrodes in which the number of unit electrodes per sheet, and the intervals of adjacent electrode terminals are varied are prepared, therefore, an electrocardiogram measurement can be performed on patients with every kind of body frame, ranging from child to adult.

### EXPERIMENTAL EXAMPLES

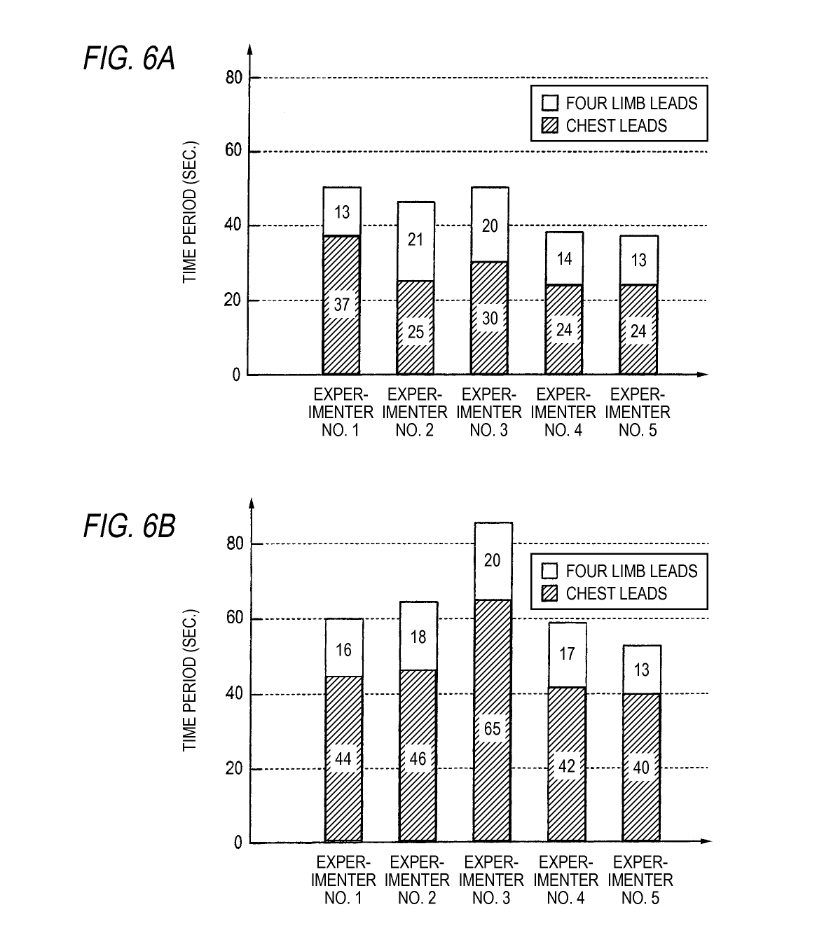

Next, experimental examples in which execution time periods when the biological electrode of the embodiment was applied were measured will be described with reference to FIGS. 6A and 6B. FIG. 6A is a view showing execution time periods when experiments in which the biological electrode of the first embodiment of the presently disclosed subject matter was applied to the subject were executed, and FIG. 6B is a view showing execution time periods when experiments in which related-art biological electrodes were applied to the subject were executed, as comparison examples. The related-art biological electrodes were produced one by one, and a predetermined number of the electrodes were packed in a bag or the like. In FIGS. 6A and 6B, the ordinate indicates the time period (sec.), and the abscissa indicates five experimenters.

As shown in FIG. 6A, execution time periods in the case where the biological electrode 10 of the embodiment was used were measured. The execution time period (average value±standard deviation) of chest leads V2 to V6 was 28.0±2.5 sec., and the total execution time period of chest leads and four limb leads was 44.2±2.8 sec. In the case of the related-art biological electrode, as shown in FIG. 6B, by contrast, the execution time period (average value±standard deviation) of chest leads V2 to V6 was 47.4±4.5 sec., and the total execution time period of chest leads and four limb leads was 64.2±5.5 sec.

In the case where the biological electrode 10 of the embodiment was used, namely, the execution time periods were shortened by an average of about 41% as compared with the case where the related-art biological electrode was used.

### Second Embodiment

In the above, the first embodiment in which unit electrodes to be used in one electrocardiogram measurement are included in one sheet of biological electrode has been described. A second embodiment of the presently disclosed subject matter in which unit electrodes to be used in a plurality of electrocardiogram measurements are included in a roll of biological electrode will be described.

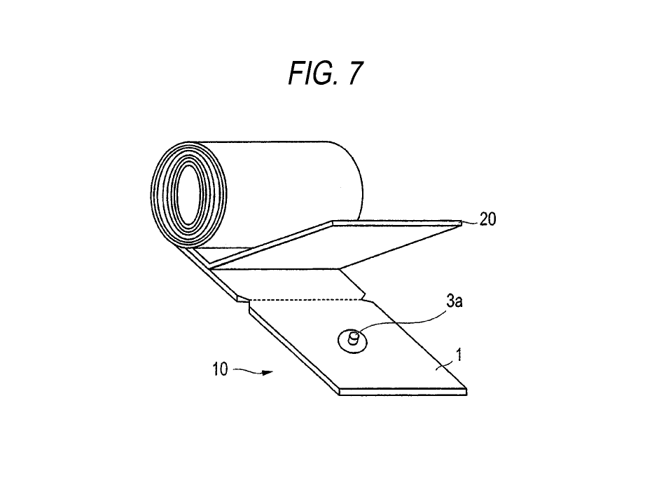

FIG. 7 is a view illustrating the roll of biological electrode of the second embodiment of the presently disclosed subject matter. As shown in FIG. 7, the roll of biological electrode 30 of the embodiment has the biological electrode 10 and a cushion member 20. In the second embodiment, unlike the first embodiment, the biological electrode 10 includes unit electrodes to be used in a plurality of electrocardiogram measurements, and is wound in roll form.

The cushion member 20 prevents the substrate 1 and conductive members of the biological electrode 10 which is wound in roll form, from being damaged by the electrode terminals. The cushion member 20 is formed by using a material such as polyethylene on the biological electrode 10 so as to be easily peeled off from the biological electrode 10, and wound in roll form together with the biological electrode 10.

In the embodiment, the biological electrode 10 is wound in roll form, and therefore a medical person can separate a series of unit electrodes the number of which is required for an electrocardiogram measurement, from the body of the roll of biological electrode 30, and then use the unit electrodes. Even when various biological electrodes in which the number of unit electrodes per sheet is varied are not previously prepared, therefore, a required number of unit electrodes can be separated and then used. As a result, the embodiment can be employed also in monitoring of the three-electrode method, or an electrocardiogram measurement in which four to six unit electrodes are used.

In the roll of biological electrode 30 of the embodiment, a plurality of unit electrodes are continuously formed in one row and wound in roll form. As compared with the case where biological electrodes are produced one by one and then packed into a bag, or the case where unit electrodes are placed respectively at the apexes of a triangular, therefore, the roll of biological electrode is not bulky, and hence has a high accommodation property. As a result, the packing material WP can be reduced in size, and the roll of biological electrode can be easily taken out from the packing material WP.

The cushion member 20 may function also as the release sheet 2. In the case where the substrate 1 and the like of the biological electrode 10 are formed by a material which is hardly damaged, the cushion member 20 may be omitted.

Although the biological electrode and roll of biological electrode of the presently disclosed subject matter have been described with reference to the embodiments, it is a matter of course that those skilled in the art can adequately perform addition, modification, and deletion within the scope of the technical concept of the presently disclosed subject matter.

The first and second embodiments in which the substrate is approximately rectangular, and also the substrate of each unit electrode is formed into a rectangular have been described. However, the substrate of each unit electrode may be formed into a shape other than a rectangle, such as a circle, an ellipse, or a polygon. Namely, substrates of unit electrodes and having a circle, an ellipse, a polygon, or the like are coupled to each other to form the substrate into a strip-like shape.

The first embodiment in which the adhesive member is disposed on the back surface of the substrate of the biological electrode has been described. However, the structure of the biological electrode is not limited to the above-described one. For example, electrically conductive adhesive gel which functions as the conductive members may be disposed over the whole back surface of the substrate, and function as the conductive member and the adhesive member. Alternatively, electrically conductive adhesive gel may be disposed in a part of the back surface of the substrate including all the base portions of the electrode terminals, an adhesive agent may be disposed on the back surface of the substrate (an adhesive tape-like member is used as the substrate), and the adhesive member may be omitted.

The first embodiment in which the biological electrode is applied to the patient and then the signal line cable is connected to the electrode terminals of the biological electrode has been described. Alternatively, the signal line cable extending from the electrocardiograph may be previously connected to the electrode terminals before the biological electrode is applied to the patient. In the alternative, preparation for an electrocardiogram measurement has been made, and therefore the electrocardiogram measurement is rapidly started after the biological electrode is applied to the patient.

According to an aspect of the presently disclosed subject matter, a plurality of unit electrodes are continuously formed in one row while being separated at equal intervals, so as to correspond to electrode application positions of the chest region of the patient. Therefore, unit electrodes which are to be separated and then applied can be reduced in number. Consequently, the biological electrode can be used in an electrocardiogram measurement requiring a large number of electrodes, and applied to electrode application positions of the patient for a short time period.

According to an aspect of the presently disclosed subject matter, a biological electrode is wound in roll form, and therefore a medical person can separate a biological electrode including the number of unit electrodes which is required in an electrocardiogram measurement, from the body of the roll of biological electrode, and then use them.

What is claimed is:

1. A biological electrode comprising:
a substrate which is insulative and flexible;
a plurality of electrode terminals which are arranged on a first surface of the substrate, and which are separated from each other at equal intervals and are arranged in one row; and
a plurality of electrically conductive members which are arranged on first portions of a second surface of the substrate, and which are not electrically conductive to each other, the plurality of electrically conductive members corresponding to the plurality of electrode terminals, the electrically conductive members each of which is connected to a corresponding one of the electrode terminals and is adapted to be electrically contactable to a living body, wherein
the substrate is formed with first separating portions between respective adjacent pairs of sets of one of the electrode terminals and a corresponding one of the electrically conductive members.

2. The biological electrode according to claim 1, further comprising adhesive members used for causing the biological electrode to adhere to the living body, which are arranged on second portions of the second surface of the substrate, and which are formed with second separating portions at positions corresponding to positions of the first separating portions.

3. The biological electrode according to claim 1, wherein the substrate is formed with notches at positions where long sides of the substrate intersect with the first separating portions, respectively.

4. The biological electrode according to claim 1, wherein the interval between adjacent two of the electrode terminals is 3 to 8 cm.

5. A roll of biological electrode which includes the biological electrode according to claim 1 which is wound in roll form.
